(19) United States
(12) Patent Application Publication
(10) Pub. No.: US 2008/0319435 A1
(43) Pub. Date: Dec. 25, 2008

(54) SHAPE-CHANGING TISSUE CONSTRICTOR AND METHODS OF USE

(75) Inventors: Robert J. Rioux, Ashland, MA (US); Jeffrey V. Bean, Fitchburg, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: BOSTON SCIENTIFIC SCIMED, INC., Maple Grove, MN (US)

(21) Appl. No.: 11/871,940

(22) Filed: Oct. 12, 2007

Related U.S. Application Data
(60) Provisional application No. 60/851,592, filed on Oct. 12, 2006.

(51) Int. Cl. A61B 17/128; A61B 17/125; A61B 17/122

(52) U.S. Cl. 606/33; 606/157; 606/139

(57) ABSTRACT

A tissue constrictor includes one or more shape-changing materials that when activated aids in constricting a portion of an organ or tissue in the body. In one embodiment, the constrictor conducts electrosurgical or ablation energy to section tissue.

### CROSS-REFERENCE TO RELATED APPLICATION

This application claims the benefit of U.S. Provisional Application No. 60/851,592, filed on Oct. 12, 2006, the disclosure of which is expressly incorporated by reference.

### FIELD OF THE INVENTION

The invention relates to medical devices in general and, in particular, to devices for constricting tissue.

### BACKGROUND

Obesity, especially morbid obesity, is associated with substantial mental and physical health risks such as diabetes, high blood pressure, and shortened life expectancy. Typical treatments for obesity include dietary restrictions and increased exercise and often have poor long-term success. Surgical options for the treatment of obesity, frequently restricted to morbid obesity, may include gastric bypass surgery, small bowel bypass surgery, and reduction of stomach volume by surgery (e.g., stomach stapling). While effective for some patients, these procedures may have unsatisfactory long-term results and may also cause other negative health effects. Less traumatic techniques for reducing available stomach volume have included balloons placed in the stomach.

Another method for treating obesity is to place a constrictive band around the exterior of the stomach to effectively separate the stomach into a small upper region near the esophagus and a larger lower region of the stomach beyond the constriction. These two portions are separated by a stoma, i.e., a small opening and surrounding tissue that is created by the constrictive band. Gastric bands retard movement of food from the upper stomach region to the lower stomach region as a result of the food having to pass through the restriction of the newly created stoma. With the gastric band in place, the patient should feel sated with less food as a result.

Adjustable gastric bands have been developed allowing post-operative resizing of the open area of the stoma with minimally invasive procedures. Adjustment may be accomplished using an inflatable reservoir for a gas or, more typically, a saline solution. Frequently, an access port is placed just under the skin during the gastric banding procedure. The access port is connected to the adjustable gastric band by tubing, thereby allowing inflation of an inflatable reservoir in the band by an introduction of gas or fluid with a syringe and needle inserted through the skin and into the access port. As the reservoir fills, it expands and compresses the stoma tissue inwardly, thereby reducing the open area of the stoma. The reservoir may also be drained or vented to increase the open area of the stoma.

Modern laparoscopic procedures are typically used to place gastric bands either with or without a calibration device into the stomach through the esophagus for determining the inner diameter and placement of the gastric band being implanted. Placement of a gastric band usually requires sectioning of tissue in the retrogastric space to provide room for the band to encircle the fundus, i.e., a “retrogastric tunnel.” Many moderm gastric band procedures employ the Kuzmak technique, wherein the retrogastric tunnel passes posterior to the stomach from the lesser curvature of the stomach, about 1 cm below the gastroesophageal junction, to the angle of His on the greater curvature of the stomach. For devices with an access port tethered to the gastric band by tubing, the port is typically also inserted during the same procedure.

Laparoscopic procedures can require the placement of five to seven cannulae, typically six cannulae, into the abdomen of the patient, each cannula opening typically being between 5 mm and 30 mm in diameter. Therefore, the placement of a gastric band can be a fairly invasive procedure that requires a substantial recovery time. Given these problems, there is a need for a mechanism that simplifies the placement of a tissue constrictor in a patient.

### SUMMARY

To address the above-mentioned concerns, several embodiments of the present invention are directed to tissue constrictors that include a shape-changing component(s) or material(s). In some embodiments, the constrictor is used as a gastric band that curves around the stomach to facilitate its deployment. In another embodiments, the tissue constrictor may include one or more position stabilizers, for example, spurs or suture points for securing the band. Additional embodiments of the present invention are adapted to section tissue, such as by delivery of high-frequency electrosurgical energy through the band. Further embodiments of the present invention are directed to delivery tools having a shape-changing component(s) or material(s). The delivery tools are used to properly place a tissue constrictor in-vivo.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### BRIEF DESCRIPTION OF THE DRAWINGS

The foregoing aspects and many of the attendant advantages of this invention will become more readily appreciated by reference to the following detailed description, when taken in conjunction with the accompanying drawings, wherein:

FIG. 1A illustrates a typical number and placement of entry points into the abdominal cavity of a patient undergoing bariatric surgery for placement of a gastric band device;

FIG. 1B illustrates a conventional placement of a retrogastric tunnel for placement of a gastric band around the stomach of a patient;

FIG. 1C illustrates a conventional tool placed in a retrogastric tunnel for aiding in the placement of a gastric band;

FIG. 1D illustrates a conventional hydraulically or pneumatically adjustable gastric band separating a stomach into an upper and lower region by creating a stoma between the upper and lower stomach regions;

FIG. 1E illustrates a conventional placement location for an access port used to adjust the volume or pressure in a hydraulically or pneumatically adjusted gastric band;

FIGS. 2A-2D illustrate one embodiment of a tissue constrictor in accordance with the present invention;

FIGS. 3A-3B illustrate another embodiment of a tissue constrictor in accordance with the present invention;

FIGS. 4A-4B illustrate another embodiment of a tissue constrictor in accordance with the present invention;

FIGS. 5A-5B illustrate another embodiment of a tissue constrictor in accordance with the present invention;

FIGS. 6A-6C illustrate another embodiment of a tissue constrictor in accordance with the present invention;

FIG. 7 illustrates one or more position stabilizers included in a tissue constrictor in accordance with another embodiment of the invention; and

FIG. 8 illustrates a system for supplying activation energy to a tissue constrictor in accordance with one embodiment of the present invention.

### DETAILED DESCRIPTION

Embodiments of the present invention will now be described with reference to the accompanying drawings where like numerals correspond to like elements. Embodiments of the present invention are generally directed to tissue constrictors for use in compressing a patient's tissue or organs. The following description depicts the primary use of the tissue constrictors as a gastric band. However, other suitable uses for constricting other tissues, vessels, or organs in the body are contemplated to be within the scope of the present invention, as claimed.

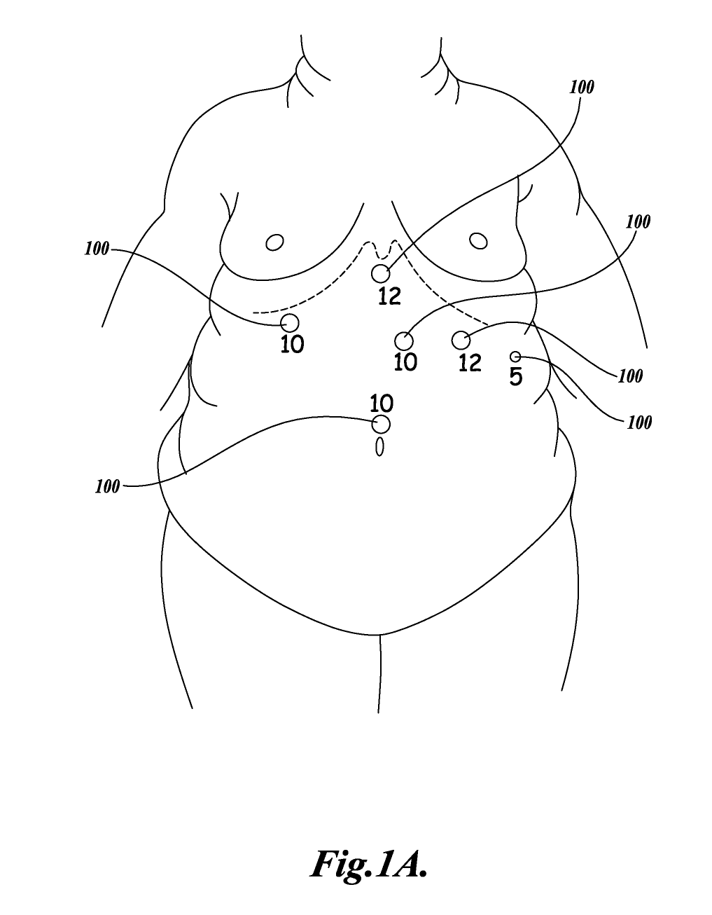

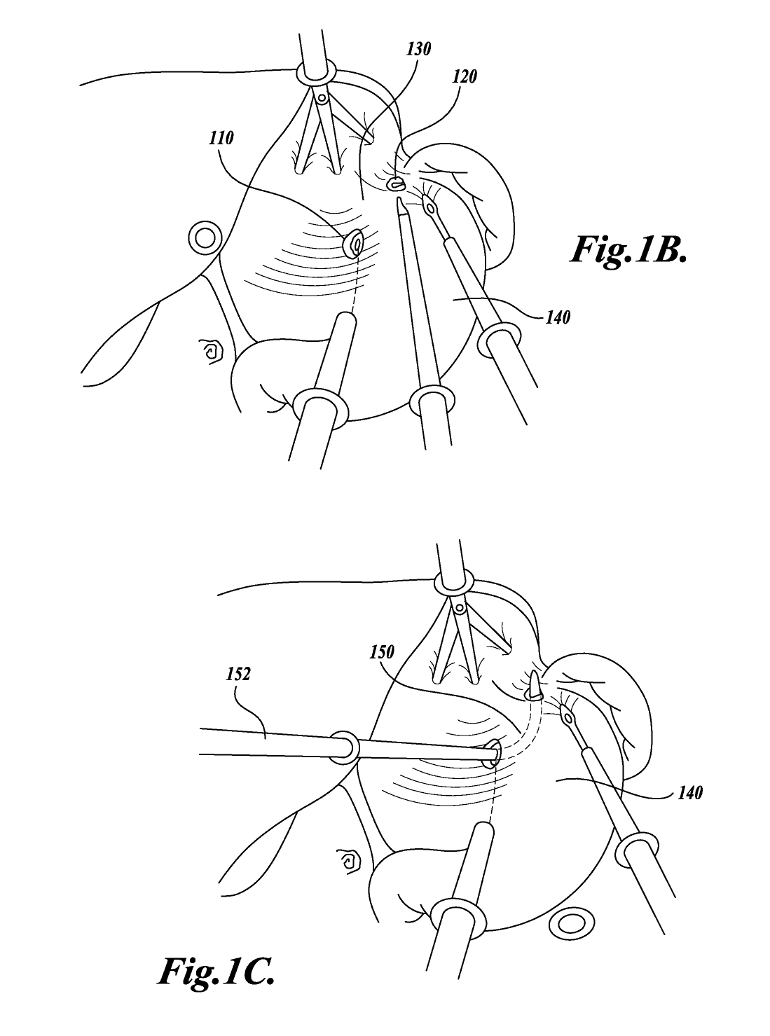

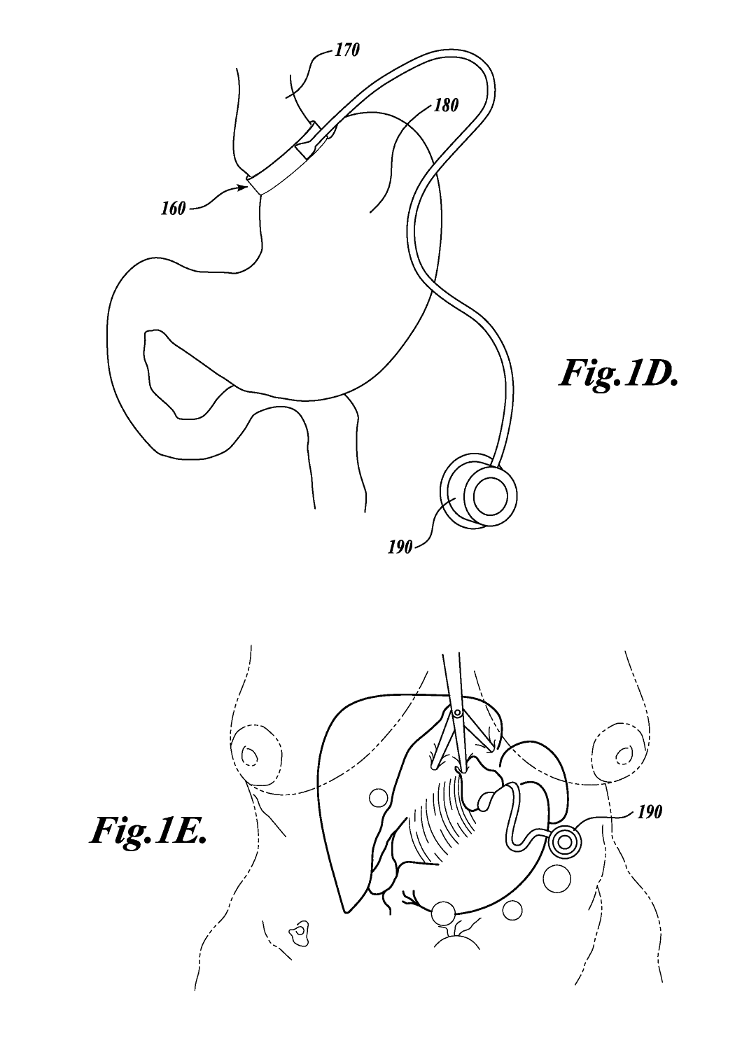

Bariatric surgery for placement of a gastric band typically includes the placement of six entry points into the abdomen of a patient, as shown in FIG. 1A. These six entry points 100 are usually formed with trochars and cannulae that are between 5 mm and 30 mm in diameter to permit, among other actions, insufflation, observation, retraction, and manipulation of tissue, sectioning of tissue, placement of a gastric band, and providing an optional access port. Once the abdominal cavity is accessed, a retrogastric tunnel may be formed in the connective tissue around the outer surface of the stomach to accommodate the gastric band.

The creation of the retrogastric tunnel may be performed by a technique that includes the creation of two tunnels. As shown in FIG. 1B, one tunnel 110 extends from the lesser curvature of the stomach and one tunnel 120 extends from the greater curvature of the stomach. The tunnels join posterior to, and slightly below, the point of the gastroesophageal junction 130 and the stomach 140.

Once the first and second tunnels are created and joined together around the stomach, an instrument 152 is inserted through the connected retrogastric tunnel 150 to pull a gastric band through the tunnel posterior to the stomach organ 140, as shown in FIG. 1C. A gastric band 160 may then be inserted through the tunnel 150 and closed around the stomach organ 140, separating the stomach 140 into an upper region 170 and a lower region 180, as shown in FIG. 1D. Constriction of the gastric band 160 creates a stoma between the two regions 170 and 180. Where the gastric band 160 is adjustable and includes an access port 190, the port is typically attached to the adjustable gastric band 160 by tubing. As shown in FIG. 1E, the access port 190 is often placed just under the skin near the front, left, lower rib cage to allow easy access and palpation for adjustment of the gastric band by injection of a fluid or gas.

As will be appreciated, the conventional technique for placing a gastric band in a patient is relatively invasive. Some embodiments hereinafter described aid the deployment of a gastric band around the stomach during bariatric surgery, potentially simplifying the surgery.

As will be described in further detail below, some embodiments of the tissue constrictor are suitable for use as a gastric band that incorporates a shape-changing material. The shape-changing material facilitates deployment of the band during bariatric surgery. Shape-changing materials include but are not limited to materials that have a “memory” shape. Some shape-changing materials can be deformed and will return to, or near to, the “memory” shape upon application of a trigger signal, such as an activating energy (electricity, heat, light, chemical, etc). Shape-changing materials include, but are not limited to, shape-changing metals or metal alloys and shape-changing plastics. One example of a suitable shape-changing metal is Nitinol™. Other suitable shape-changing materials include, but are not limited to, thermoplastics such as shape memory polyurethanes, cross-linked trans-polyoctylene rubber, polynorbornene polymers and copolymers and blends thereof with styrene elastomer copolymers, such as Kraton, and polyethylene; styrene butadiene copolymers; PMMA; polyurethane; cross-linked polyethylene; cross-linked polyisoprene; polycycloocetene; bioabsorbable shape memory polymers such as polycaprolactone, copolymers of (oligo)caprolactone, PLLA, PL/DL A copolymers, and/or PLLA PGA copolymers; and Azo-dyes and/or Zwitterionic and/or other photochromatic materials such as those described in “Shape Memory Materials” by Otsuka and Wayman, Cambridge University Press (1998), the entire contents of which are being incorporated herein by reference.

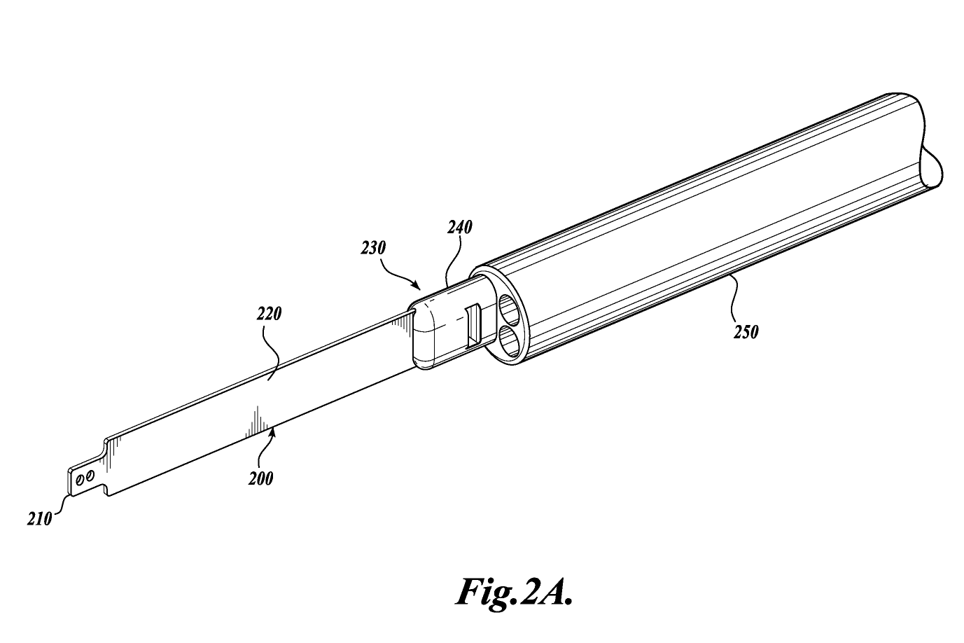

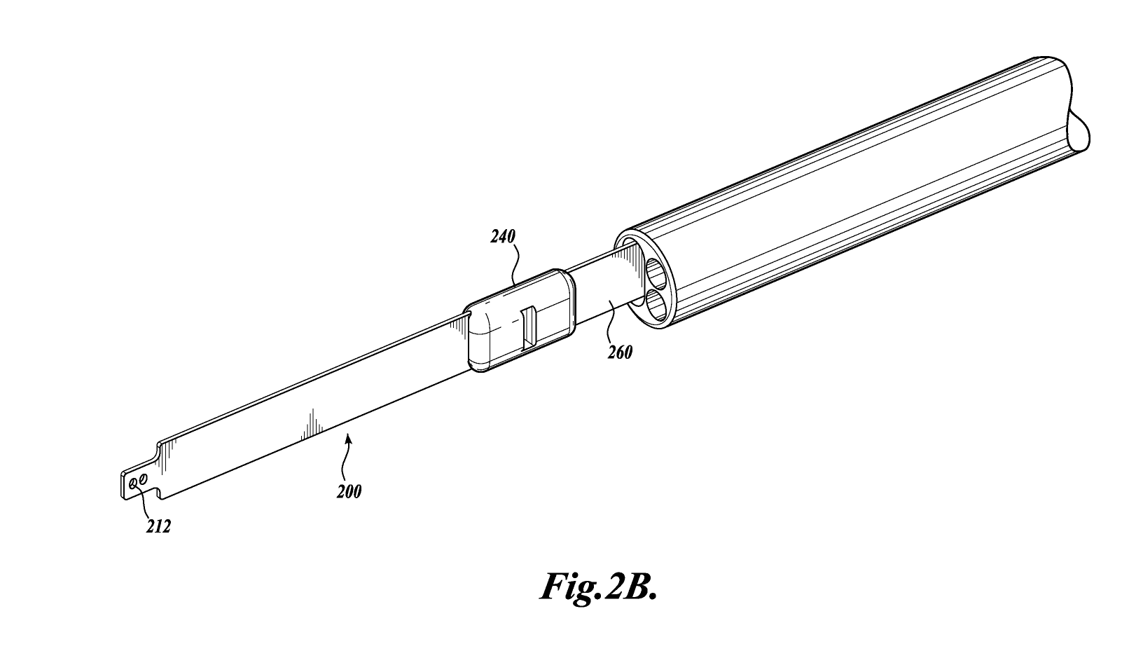

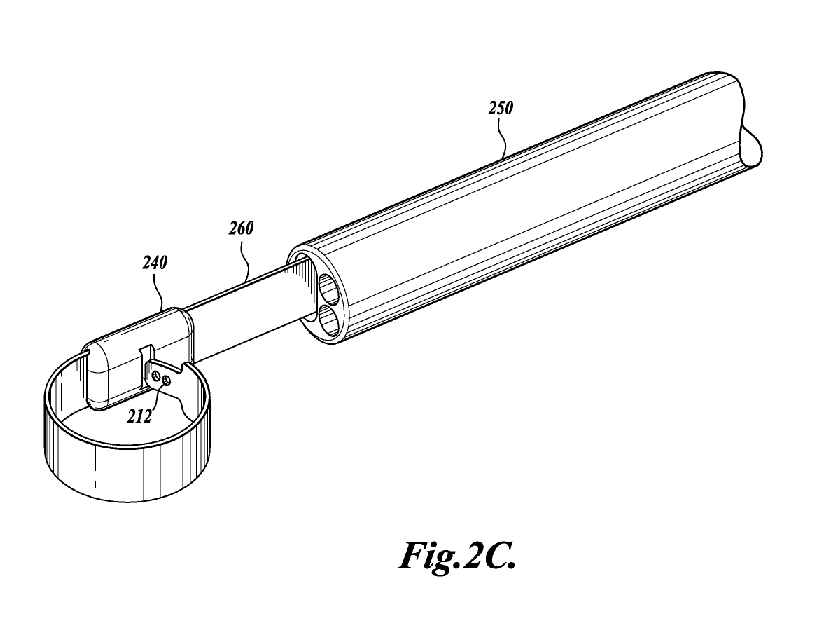

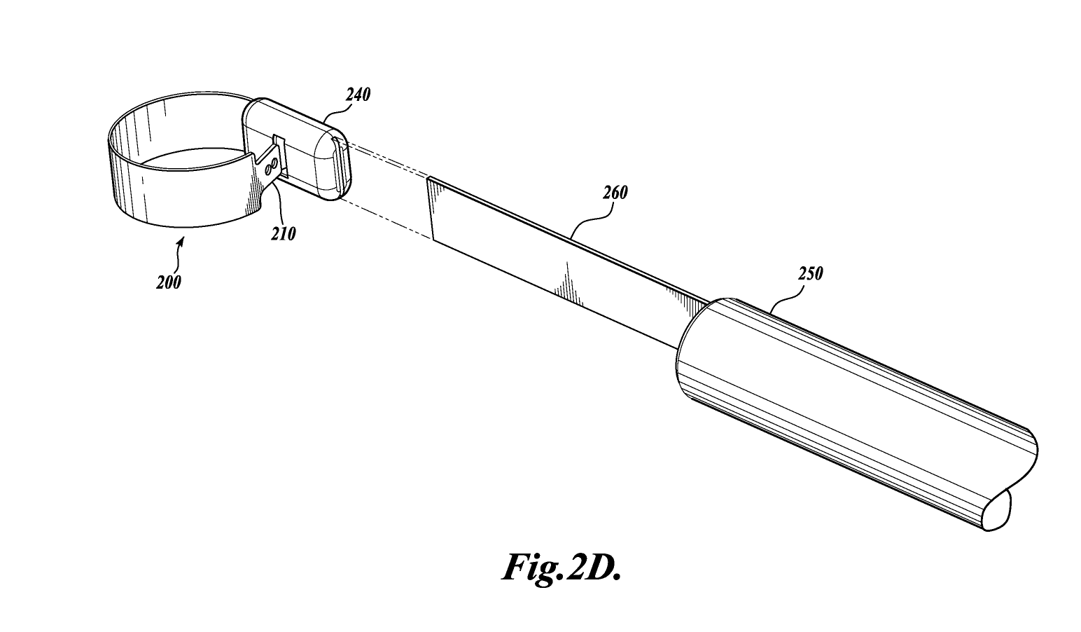

FIGS. 2A-2D illustrate one embodiment of a tissue constrictor formed in accordance with aspects of the present invention. The tissue constrictor, such as a gastric band 200, is positioned at a desired location in the body via a delivery catheter 250. The delivery catheter 250 may be routed through an additional catheter, endoscope, a trocar, a dilator, an introducer, a needle with an axial bore, a hollow tubing, a cannula, or the like. Some bariatric procedures may obviate the need for the additional introducer, for example, during open bariatric surgery, as opposed to laparoscopic or endoscopic procedures, whereby the abdomen is directly accessed through one or more incisions. Therefore, where an access device is inappropriate or unnecessary, the gastric band 200 may be employed without an additional access device.

In the embodiment shown, the gastric band 200 includes a distal tip 210, a central band portion 220 extending proximally from the distal tip, and a proximal end 230. A receiver 240 positioned at the proximal end 230 of the band 220 has a tip mating mechanism that engages and secures the tip 210 to hold the band 200 around a desired object such as a portion of the stomach. In one embodiment, the proximal end 230 of the gastric band 200 is removably attached to the distal end of a delivery tool 260 that is routed in the delivery catheter 250 as shown in FIG. 2B. The gastric band 200 may be inserted into the abdominal cavity of a patient through an access device while attached to the distal end of the delivery tool 260. Once in place, the gastric band 200 is activated to bend the gastric band at least partially around the stomach, as will be described in further detail below.

The gastric band 200 may be comprised of a single type of shape-changing material such as Nitinol™. Alternatively, the gastric band 200 may comprise a plurality of materials, for example, as laminated layers of structurally different shape-changing materials, or the same shape-changing material having different memory shapes. Alternatively, one or more shape-changing materials may be combined with non-shape-changing materials.

In one embodiment, the materials used to construct the gastric band 200 are selected so that, upon activation, the gastric band will change to its memory shape when placed in the body (e.g., temperature activated).

FIGS. 2C and 2D illustrate one possible “memory” state shape of the gastric band 200. After the shape-changing material of the gastric band is actuated, the gastric band 200 transitions to the memory state shape. For example, if the memory state shape is as depicted in FIG. 2C, the gastric band 200 attempts to return to a generally closed shape by bending into a circular configuration such that the distal tip 210 is in contact with the receiver 240 (or nearly so) at the proximal end 230 of the band. In the embodiment shown, the distal tip 210 of the band 200 includes a key 212 having one or more holes 214 therein. The key 212 at the distal tip is sized to fit within a slot 242 in the receiver 240. The receiver 240 includes a locking mechanism such as one or more pins that engage the holes 214 to secure the tip 210 in the receiver 240. The tip may be fixedly secured or releasably secured in the receiver 240. For example, the pins may be spring biased in order to allow them to be removed from the holes 214 so that the tip 210 can be withdrawn from the receiver 240.

In some embodiments, a spring force is associated with the materials used to form a gastric band 200. As a gastric band 200 attempts to return to its “memory” state shape upon actuation, this spring force may be exerted on tissue structures that are positioned within the closing band structure. If the spring forces are not sufficient to overcome the resistance imposed by these interfering structures, the gastric band 200 may be prevented from attaining the final “memory” state shape. Therefore, in one embodiment, the gastric band 200 is designed with the correct geometry, materials, and size to accomplish a complete or nearly complete closure in order to create a desired constriction of tissue or organ around which it is placed. Where the spring force is insufficient to close the gastric band, the final closure of the band may be accomplished manually, such as with another tool or by hand.

As indicated above, once the gastric band 200 is closed, the distal tip 210 may or may not be removed from the receiver 240 at the proximal end 230 of the band. The design of the gastric band 200 may be intended for extended or permanent implantation into a patient and, thus, the distal tip 210 may be non-removably engaged with the receiver 240. Alternately, the connection between the distal tip 210 and the receiver 240 may be removable to facilitate later removal of the device from the patient.

Examples of non-removable connections between the tip portion 210 and the receiver 240 include sutures, welds, crimps, thermal glues, chemical glues or other adhesives, mechanical friction locks, hooked structures (either a hook structure in the receiver 240 that engages the distal tip 210 or a hook in distal tip 210 that engages a cooperating structure in the receiver 240), cross-pin structures, mechanical locking structures (for example zip-tie closures, key-type locks, cam locks, flexible tangs and male and female fasteners and components, cooperating slots, etc.), strong permanent or electromagnetic components, screws or bolts either perpendicular to or parallel with the distal tip 210, or rivets, among others.

Further examples of removable connections between the distal tip portion 210 and the tip mating mechanism 240 include any breakable, reversible, or actuatable connections listed above. Additionally, removable connections may, for example, include hook and loop-type materials such as Velcro™ or dissolvable sutures, among others, and may also include electrolytic joints, adhesives, etc.

In some embodiments, the proximal end 230, distal tip 210, and/or receiver 240 may be used to adjust the inner diameter of the closed gastric band 200. In one embodiment, the diameter of the gastric band is adjusted by moving the tip portion out of the receiver. This may be accomplished by passing the tip portion 210 through the receiver 240 in a manner similar to a zip-tie. Alternatively, this may be accomplished by moving the position of the receiver 240 along the band 200.

In yet another embodiment, the diameter of the gastric band can be changed by compressing or expanding the band 200 while the distal tip 210 is in the receiver 240. Changing the diameter of the gastric band 200 may be accomplished by the use of ancillary structures such as reversibly inflatable reservoirs or mechanical actuators placed between the band and the surrounding tissue.

A removable delivery tool 260 may be associated with the gastric band 200. The delivery tool 260 may be a general purpose laparoscopic or endoscopic tool well known in the art, or may be a tool specifically associated with particular embodiments of the gastric band 200. Generally, the delivery tool 260 is an elongated rod or shaft that may be used to place, manipulate, deploy, activate, or adjust, or some combination thereof, a gastric band 200 during installation, removal, or adjustment. In one embodiment, the delivery tool 260 comprises a rigid shaft or thin bar having the gastric band 200 secured at one end thereof and a length selected so that a user can move the gastric band from the proximal end of the delivery catheter 250. Alternate embodiments of the delivery tool 260 may comprise a flexible shaft, wherein the flexible shaft still permits guidance of the gastric band 200 to a desired location. Flexible shaft embodiments generally are also torsionally stiff to permit rotation of the gastric band 200 from the proximal end of the tool. In addition, the shaft may have variable stiffness or a controllable stiffness, steerability, etc.

In one embodiment, the delivery tool 260 is communicatively connected, in addition to being removably connected, to a gastric band 200. For example, the delivery tool 260 may include one or more electrical conductors to transmit electrosurgical energy to the gastric band 200. In an alternative embodiment, the delivery tool 260 comprises at least one fluid or gas conduction lumen to transmit said fluid or gas to, or near to, the gastric band 200. The transmitted fluid or gas may, for example, be used to heat or cool the gastric band 200, to inflate or deflate a reservoir, or to clean the gastric band 200, or some combination thereof, among other actions. Multiple features may be embodied in shaft and band. For example, the delivery tool 260 may include a fluid conduit and a conductor for activating the band material and/or the fluid contained therein.

In one embodiment, the delivery tool 260 includes one or more electrical conductors to transmit sufficient electrical energy to an electroresistive heating element that is thermally coupled to the gastric band in order to raise the temperature of the gastric band 200 or a component thereof to a temperature sufficient to activate the shape-changing material. A still further embodiment of a delivery tool 260 includes one or more thermally conductive elements such as copper rods or other heat conducting metals, capable of transmitting externally generated thermal energy to raise the temperature of a shape-changing material in the gastric band 200 to a level sufficient to activate the shape-changing material into its memory state. If the gastric band is made of an electrically conductive material, the actuation energy may be delivered directly to the band. If not conductive, the actuation energy may be coupled to a wire or printed circuit trace on the band that causes the band to assume its memory shape. In other embodiments, the shape-changing material may be activated by light and the actuation energy delivered from a light source by an optical fiber or light source positioned near the band.

The delivery tool 260 may be removably connected to the gastric band 200 by means of a severable link, including, for example, a thinned portion, a narrow portion, a necked portion, a perforated portion, and/or electrolytic or melting links, among others. Alternatively, the delivery tool 260 shaft may be separated from the gastric band 200 by bending, rotating, or pulling with sufficient force, or applying an energizing force, among others. The removable connection may also comprise mechanical removable connections, for example, mechanical severing devices, friction connections such as a pin and socket, screw threads, or actuatable mechanisms such as retractable hooks, among others. Additional embodiments of the removable connection may comprise permanent-magnetic or electromagnetic removable connections. Further additional embodiments of the removable connection may include a breakable adhesive connection such as a chemically or thermally weakened or destroyed adhesive connection, among others.

Once in the desired location, the gastric band 200 is separated from the delivery tool 260, as shown in FIG. 2D.

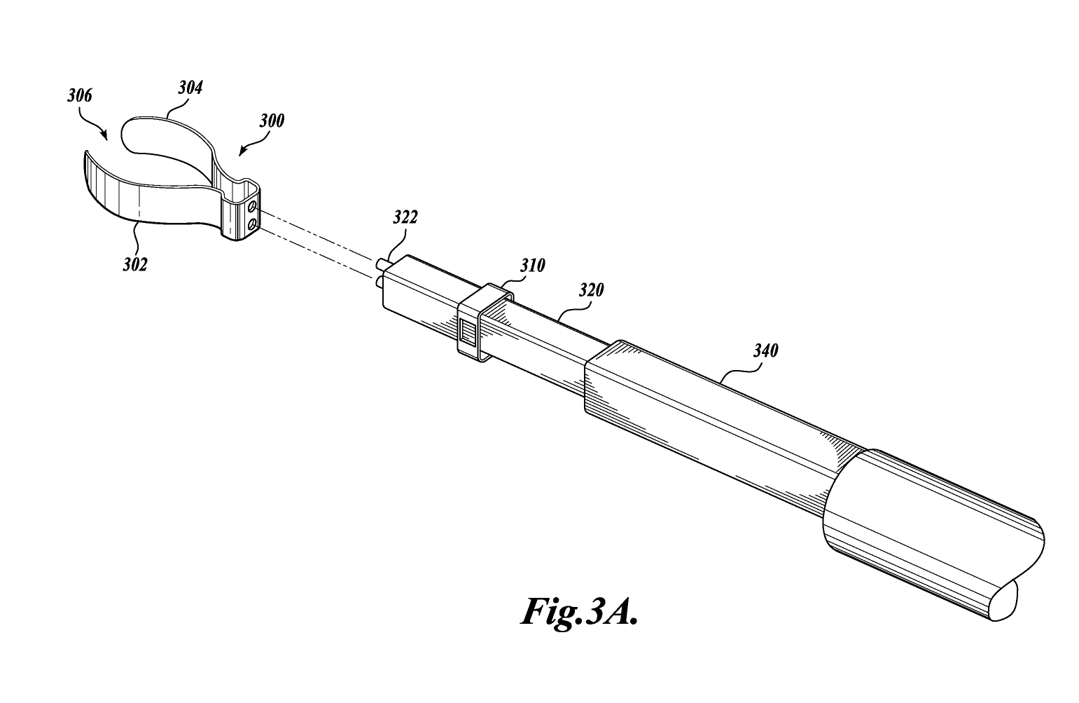

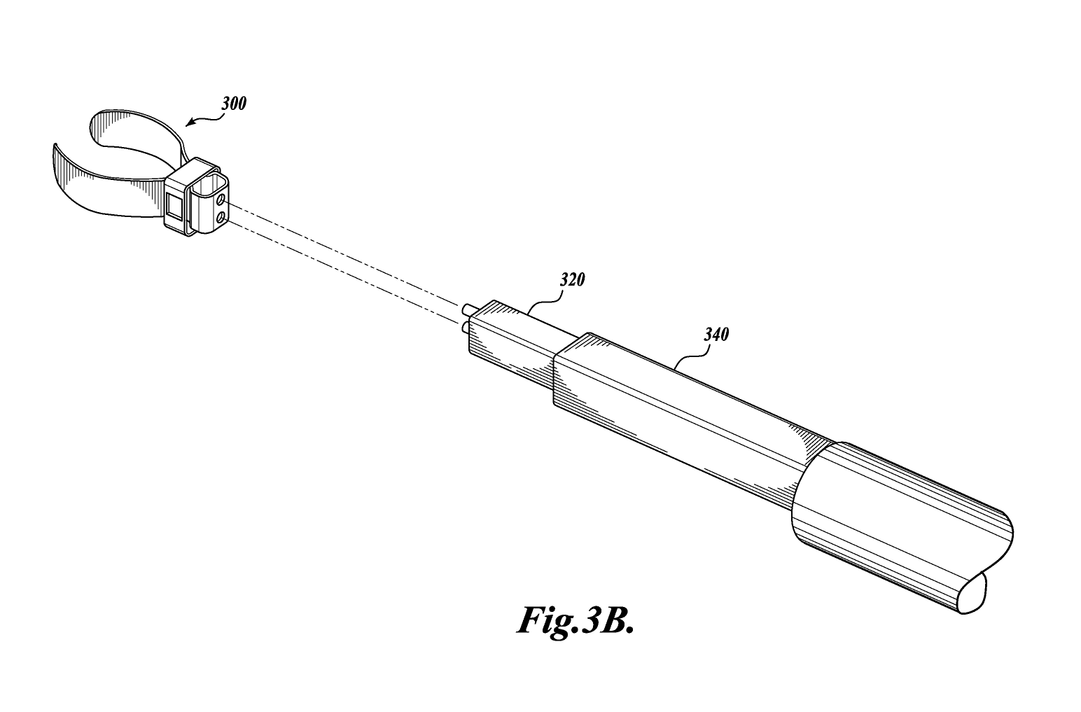

FIGS. 3A-3B depict an alternative embodiment of the present invention. In this embodiment, a tissue constrictor that may be used as a gastric band 300 has a generally “horseshoe” shape including a pair of semicircular legs 302, 304 that are joined at one end and are spaced apart by a gap 306 at their tips. A leg retaining mechanism 310 is selectively positionable over the area where the legs 302 and 304 are joined together. By moving the leg retaining mechanism 310 proximally or distally, the legs are squeezed together or are allowed to expand apart. In one embodiment, the legs 302, 304 of the gastric band are made of a shape-changing material such as Nitinol™. The distal tips of the legs 302, 304 can remain apart or may be joined together upon activation of the shape-changing material by an activation energy. Such energy may include resistive heating of the legs. Alternatively, externally generated activation energy can be delivered to the gastric band 300 through an appropriate conduit.

The gastric band 300 is connected to a delivery mechanism by a breakable link 322. In one embodiment, the breakable link includes one or more pins that are affixed in corresponding holes on the portion of the band where the legs 302 and 304 are joined. The pins are secured to the band with a friction fit or are breakable so that the application of a sufficient force will break the connection. In the embodiment shown, the delivery mechanism includes a nested pair of catheters 320 and 340. The outer catheter 340 applies distal pressure to the leg retaining mechanism 310, while the gastric band 300 is pulled proximally by the catheter 320 in order to advance the leg retaining mechanism 310 distally over the legs 302, 304, thereby compressing or holding them together. FIG. 3B illustrates the gastric band 300 freed from the delivery mechanism.

In one embodiment, the distal tips of the legs 302, 304 close around a tissue, vessel, or organ by the application of an activation energy to the shape-changing material. The tips of the legs may become engaged via a mechanical, magnetic, or an adhesive closure mechanism, or the like, in order to create a circumferential band around the tissue. In addition, the leg retaining mechanism 310 engages the outer surface of the legs 302, 304 with a friction enhancing mechanism to prevent the retaining ring from inadvertently sliding with respect to the legs so that the band remains closed with the desired tension.

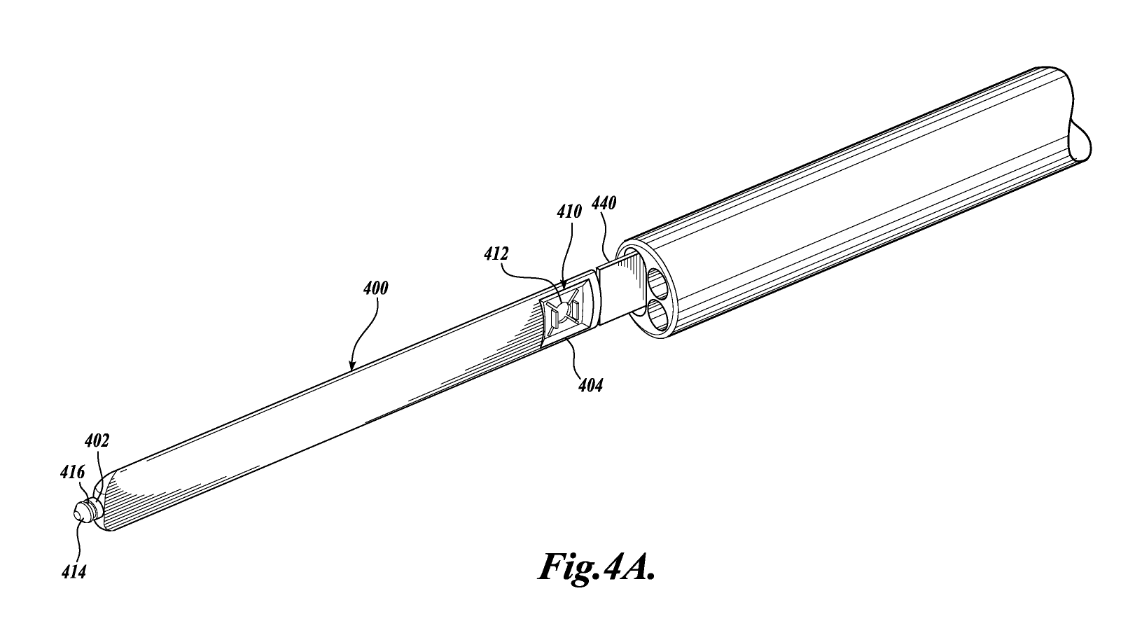

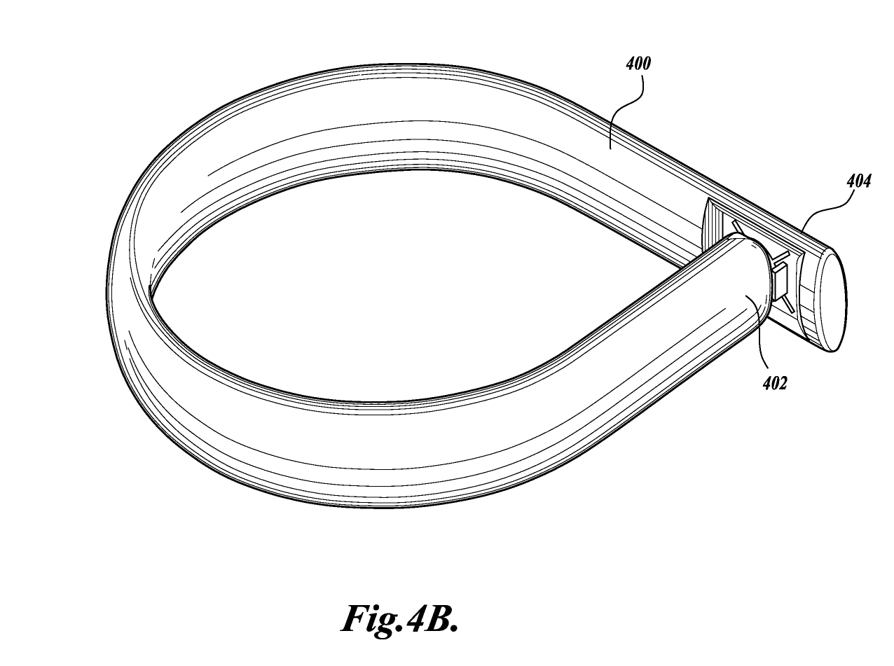

FIGS. 4A and 4B show yet another alternative embodiment of the present invention. In this embodiment, a tissue constrictor that may be used as a gastric band 400 has a distal tip 402 that is securable in a receiver mechanism 404 at the proximal end of the band. In the embodiment shown, the cross section of the gastric band between the distal tip section 402 and receiver mechanism 404 is generally curved on its inner surface so that the band smoothly engages tissue with no sharp edges and further adds pressure around the tissue when the band is closed. The curved cross section may be provided by forming the gastric band of a shape-changing plastic material, as described above. Alternatively, a shape-changing metal can be used and covered with a plastic or rubberized biocompatible material to provide the desired shape. The gastric band 400 is releasably secured to a delivery mechanism 440 that serves to place the gastric band in the patient. In addition, the delivery mechanism 440 may deliver a trigger signal, such as an activation energy, to the band in order to cause the shape-changing material to return to its memory state.

In the embodiment shown in FIGS. 4A and 4B, the receiver mechanism 404 at the proximal end of the gastric band 400 includes a window having a number of flaps 412 therein. A hole in the center of the window receives a button 414 at the distal end of the band. The button includes a groove 416 about its circumference. Upon entry of the button 414 into the window 410, the edges of one or more of the flaps 412 seat within the groove 416 to secure the distal end of the band to the proximal end as shown in FIG. 4B.

The gastric band 400 is releasably secured to a delivery mechanism 440 so that upon delivery of sufficient force or an activation energy, the gastric band 400 is released.

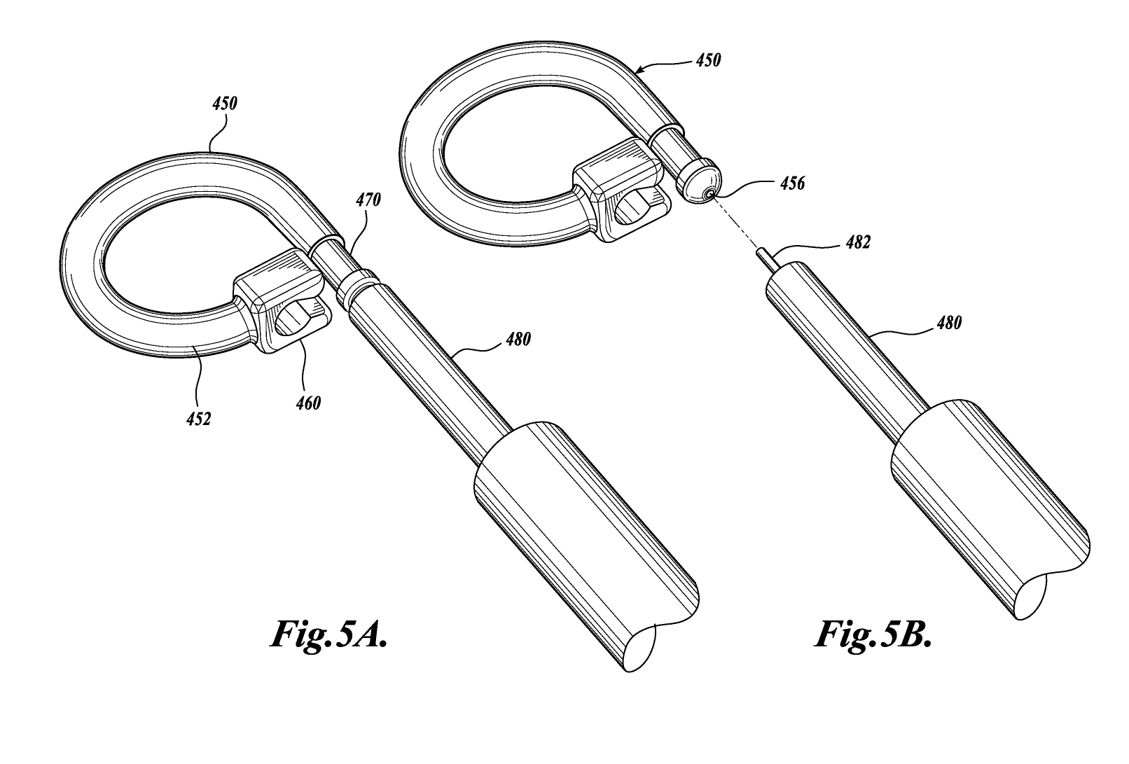

FIGS. 5A and 5B illustrate yet another embodiment of a tissue constrictor in accordance with the present invention. In this embodiment, the tissue constrictor formed as a gastric band 450 has a generally circular cross-sectional shape. A C-shaped connector 460 at the distal end of the band includes a pair of jaws that engage a corresponding post 470 positioned at the proximal end of the band. Alternatively, the C-shaped connector could be placed at the proximal end of the band and a corresponding bar or post positioned at the distal end of the band. During use, activation of a shape memory material that forms the band 450 or is included within the gastric band 450 causes the band to close completely or partially upon itself such that the distal end of the band can be secured to the proximal end of the band. In one embodiment of the invention, the gastric band 450 is coated with a polymeric or other biocompatible material such that the outer shape of the band is generally round. In an alternative embodiment of the invention, the gastric band 450 may include an outer cover that is inflatable with a gas or a liquid. Inflation of the outer cover further increases the pressure exerted by the band when it is closed. The outer cover may be further inflated or deflated to increase or decrease pressure as desired. The gastric band 450 is connected to a delivery mechanism 480 via a breakable link. As shown in FIG. 5B, the delivery mechanism 480 includes a post 482 that is received in a corresponding hole 456 at the proximal end of the band. Upon application of sufficient force or an activation energy, the post 482 is released from the hole 456, thereby releasing the band 450 from the delivery mechanism 480.

In one embodiment of the invention, the hole 456 may further be coupled to a source of air or liquid via a tube (not shown) and a reservoir secured under the patient's skin or at a similar location that allows the gastric band to be inflated or deflated as desired.

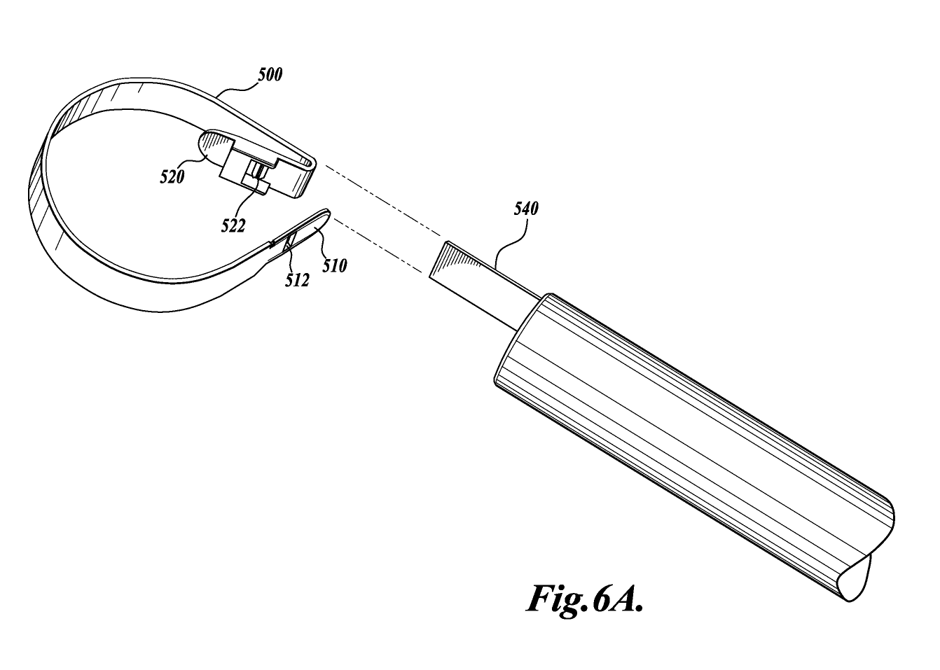

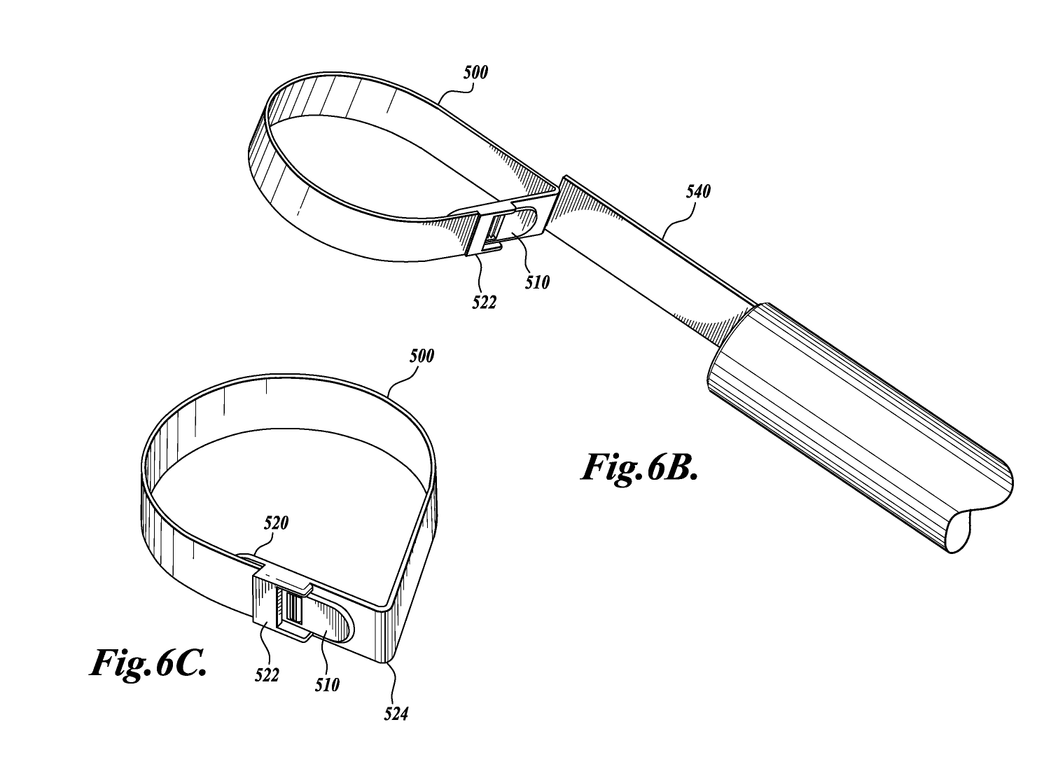

FIGS. 6A and 6B illustrate yet another alternative embodiment of a tissue constrictor in accordance with the present invention. In this embodiment, the tissue constrictor is used as a gastric band 500 having a distal tip 510 and a proximal end 520. In this embodiment, the proximal and distal ends form a cooperating tab and slot to secure the distal end to the proximal end. In the embodiment shown, the proximal end includes a loop 522 under which a tab at the distal end 510 is fitted. A pin or extension in the loop 522 fits within a slot 512 in the proximal end of the band to secure the proximal tab within the loop 522. In the embodiment shown, the proximal end 520 of the band is folded against the inner circumference of the band at a hinge 524. As shown in FIG. 6C, upon application of an activation energy, the band 500 assumes its memory state whereby the proximal end 520 is moved away from the inner circumference of the band and the distal tip 510 is moved toward the proximal end of the band. The distal tip 510 can then be fitted within the loop 522 such that the proximal end is secured to the distal tip. The gastric band 500 is delivered to a desired location with a delivery mechanism 540. Upon application of sufficient force or activation energy, a link between the delivery mechanism 540 and the gastric band 500 is broken, thereby leaving the gastric band 500 at a desired location in the patient's body.

In some embodiments of the invention, the strip that forms the band may include one or more rib structures along the band portion. The use of one or more rib shapes may provide additional rigidity and reduce or prevent twisting or rolling of the tissue constrictor after implantation in the patient. The rib shapes may be concave, convex, solid, or hollow features. The one or more rib structures may or may not be present prior to activation of a memory material comprising the gastric band. The rib structures themselves may or may not be formed out of a shape-changing material and may or may not have the same activation energy source and level as any other shape-changing material comprising the strip of the gastric band. The geometry of the rib structures may vary in number, orientation, width, length, depth, and height either as a group, including a group of one rib, or as individual ribs, where more than one rib is used.

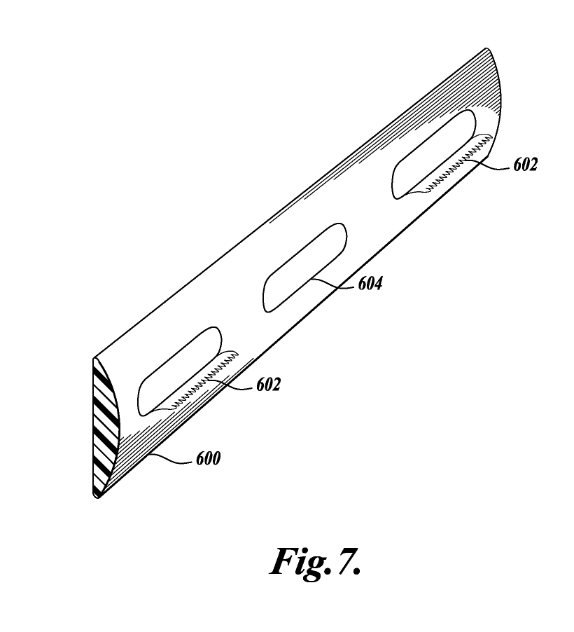

In another embodiment of the invention, the tissue constrictor may include one or more shape-changing stabilizers. The stabilizers are intended to reduce or prevent movement or twisting of a gastric band. As shown in FIG. 7, a gastric band 600 includes one or more position stabilizers that include spurs 602, anchors, or the like, that aid in fixing the position of the band with respect to the stomach or other location in the body. For example, the gastric band may include one or more holes 604 into which tissue can extend to secure the position of the band. Position stabilizers may or may not be formed of the same shape-changing material or materials as the gastric band itself. The position stabilizers may or may not include other materials in addition to a shape-changing material. In one possible embodiment, a spur-type relative position stabilizer includes a spur 602 that, upon activation, extends out of the plane of the band 600 to engage adjacent tissue in order to anchor the band. The particular angle and orientation of the spur “memory” shape state may be selected based on the intended use of the tissue constrictor.

In other embodiments, anchoring is achieved by surface structure such as a roughened surface, bumps, pits, spikes, filaments, patterns, etc. In other embodiments, the device promotes tissue growth, where permanency is desired. In another embodiment, the shape-changing component is an introduction catheter, a guide wire, or other ancillary component that bends itself around a desired structure, such as a retrogastric tunnel. The guidewire can then lead the constriction device around the tunnel for proper placement, or the constriction device can be situated on the shape-changing delivery device as it is delivered. The delivery device may create the tunnel with energy.

In some embodiments of the invention, the shape-changing materials used to form the tissue constrictor can be tailored such that the application of radio frequency energy both causes the gastric band to act as a tissue cutting device, as well as causes the gastric band to assume its memory shape, thereby surrounding the desired portion of the stomach. When used as a gastric band, the application of the radio frequency energy causes the band to tunnel around the esophageal neck above the stomach. Because the esophagus has a predictable diameter, the delivered band can be selected to have the proper predetermined diameter when closed.

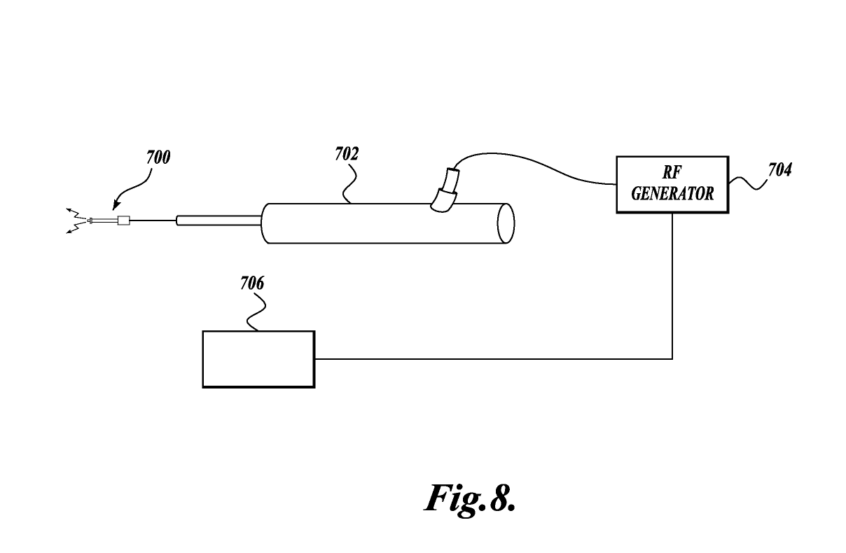

FIG. 8 illustrates one embodiment of a system for supplying the radio frequency energy to a tissue constrictor 700 that is delivered to desired location by a delivery catheter 702. A radio frequency generator 704 is in electrical contact with the tissue constrictor 700. A conductor on the tissue constrictor or the tissue constrictor itself acts as a radio frequency knife, cutting tissue surrounding the constrictor. Current is returned to the radio frequency generator 704 from an external patient pad 706. Alternatively, a return electrode may be placed in the catheter 702. The radio frequency energy delivered cuts the tissue and may activate the shape-changing material to assume its memory state. In some embodiments, the radio frequency generator may supply only an activation energy to activate the shape-changing material.

A tissue constrictor of the present invention may also include an imageable component, such as a radiopaque marker. Incorporation of an imageable component into the band may aid in placement, monitoring, or removal of such a constrictor. As an example, an embodiment with a Nitinol™ band portion is easily viewable with x-ray imaging devices because Nitinol™ is radiopaque. The imageable component may or may not be a shape-changing material, and may or may not serve any other purpose than to allow imaging of the imageable component itself. Other embodiments of the tissue constrictor includes an imageable component that is easily viewable via magnetic resonance imaging (MRI) techniques.

While the preferred embodiments of the invention have been illustrated and described, it will be appreciated that various changes can be made therein without departing from the scope of the invention. For example, although the disclosed embodiments of the invention are directed to gastric bands, the present invention could be used to constrict blood vessels, muscles, bones, internal organs, or the like. In addition, various band widths may be used to cover more surface area. More than one band could be displayed or the bands can be made easily removable.

The embodiments of the invention in which an exclusive property or privilege is claimed are defined as follows:

1. A tissue constrictor, comprising:
a band that includes at least one shape-changing material therein that upon application of an activation energy causes the band to assume a memory shape that at least partially closes the band around a portion of a patient's body.

2. The tissue constrictor of claim 1, wherein the band includes a proximal end, a distal end, and a receiver that secures the proximal end to the distal end.

3. The tissue constrictor of claim 1, wherein the band includes a cover that is selectively inflatable to increase or decrease pressure on the patient's body exerted by the constrictor.

4. The tissue constrictor of claim 1, wherein the band includes a pair of legs that are joined at one end and are open at another end and a shape-changing material having a memory shape that, when activated, causes the pair of legs to compress together.

5. The tissue constrictor of claim 4, further comprising a ring selectively positionable over the two legs to maintain the position of the legs.

6. A gastric band for apportioning a stomach into an upper and a lower region separated by a stoma having an open area between the upper and lower stomach regions as a result of constricting the stomach organ with the gastric band, the gastric band comprising:
a body including a proximal end and a distal end;
wherein the body includes at least one shape-changing material that assumes a memory state such that the distal end of the body moves toward the proximal end of the body when activated to apportion the stomach into an upper and lower a region.

7. The gastric band of claim 6, wherein the at least one shape-changing material is selected from the group consisting of a shape-memory metal, a shape-memory metal alloy, and a shape-memory plastic.

8. The gastric band of claim 6, wherein the body includes two or more shape-changing materials, and wherein the two or more shape-changing materials are selected from the group consisting of a shape-memory metal, a shape-memory metal alloy, a shape-memory plastic, and combinations thereof.

9. The gastric band of claim 8, wherein the two of more shape-changing materials have different memory states and/or different activation energies.

10. The gastric band of claim 6, wherein the body includes one or more position stabilizers.

11. The gastric band of claim 10, wherein the one or more position stabilizers include one or more spurs projecting outwardly from the body for engaging tissue and/or one or more holes in the body for receiving tissue therein.

12. The gastric band of claim 6, further comprising means for anchoring the body in-situ.

13. The gastric band of claim 6, wherein the body includes an imageable component that is visible with an external imaging system.

14. The gastric band of claim 6, wherein the memory state of the shape-changing material comprises either a partially closed shape or a closed shape.

15. The gastric band of claim 6, further comprising a means for adjusting the diameter of the closed band.

16. The gastric band of claim 6, wherein the body is communicatively coupled to a source of activation energy.

17. The gastric band of claim 16, wherein the activation energy is selected from a group consisting of electricity, heat, light, radiation, and a chemical.

18. The gastric band of claim 6, further comprises a conductor associated with the body, the conductor being coupled to a source of radio frequency energy that allows the conductor to cut through a patient's tissue

19. A method of applying a gastric band around the stomach of a patient, comprising:
creating a passageway around the stomach of a patient;
inserting a gastric band that includes a distal-end, a proximal end, and a shape-changing material therein into the passageway;
activating the gastric band so that the gastric band assumes a memory shape whereby the gastric band at least partially closes around the stomach.

20. An assembly, comprising:
a tissue constrictor; and
a delivery tool for delivering the tissue constrictor to a selected position in-vivo;
wherein either the tissue constrictor or the delivery tool includes a shape changing component that aids in the positioning of the tissue constrictor with respect to a selected body tissue or organ when in-vivo.

21. The assembly of claim 20, wherein the delivery tool is selected from a group consisting of a catheter, an endoscope, a trocar, a dilator, an introducer, a needle with an axial bore, a hollow tubing, a cannula, and a guide wire.

22. The assembly of claim 20, wherein the tissue constrictor is removably coupled to the delivery tool.

23. The assembly of claim 20, wherein the tissue constrictor includes a band having a proximal end and a distal end, and a connector that couples the proximal end to the distal end.
